(19) United States
(12) Patent Application Publication
(10) Pub. No.: US 2019/0022153 A1
(43) Pub. Date: Jan. 24, 2019

(54) A LIQUID PROBIOTIC COMPOSITION STABLE AT AMBIENT TEMPERATURE

(71) Applicant: Synergia Life Sciences Pvt. Ltd, Mumbai (IN)

(72) Inventors: Dilip S. Mehta, Mumbai (IN); Anselm De Souza, Mumbai (IN)

(21) Appl. No.: 16/069,812

(22) PCT Filed: Jan. 17, 2017
(86) PCT No.: PCT/IB2017/050232

§ 371 Date: Jul. 12, 2018

(30) Foreign Application Priority Data
Jan. 19, 2016 (IN) 201621001880

(51) Int. Cl. A61K 35/742; A61K 35/747; A61K 36/064; A23L 33/135

(52) U.S. Cl. CPC A61K 35/742; A61K 35/747; A61K 36/064; A23L 33/135; A23V 2250/6422; A23V 2250/762; A23V 2250/6418; A23V 2250/642; A23V 2002/00

(57) ABSTRACT

The present invention provides a stable liquid syrup composition compositions comprising probiotic bacteria, retaining their cellular viability at ambient storage conditions for at least two years.

### RELATED APPLICATION

This application claims priority from provisional application number 201621001880 dated 19 Jan. 2016 filed with Mumbai Patent Office (India) and is incorporated herein in its entirety.

### FIELD OF INVENTION

The present invention relates to stable liquid syrup compositions comprising probiotic bacteria, retaining their cellular viability at ambient storage conditions for at least two years.

More particularly the invention relates to stable liquid syrup compositions comprising probiotic bacteria belonging to the genera bacillus/lactobacillus which retain at least 75% of the activity when stored at ambient storage conditions for subsequent use as probiotic syrup, food, food supplement, and pharmaceutical compositions.

### BACKGROUND OF THE INVENTION

Probiotics can be termed as “live microorganisms” which when consumed in adequate amounts confer a health effect on the host. Probiotics today find applications in diverse fields such as food, meal replacements, dietary supplements, nutraceuticals, over the counter drugs as well as prescription drugs. The most widely marketed probiotics are dairy products and probiotic-fortified food. The products have to be stored at low temperatures to maintain the probiotic activity and have a relatively shorter shelf life. Further Probiotics can grow in these products using carbon and nitrogen sources and ferment the products giving rise to offensive odour to the products. In other fields tablets, capsules, and sachets containing the bacteria in freeze-dried form are more common. Probiotics play an important role in immunological, digestive and respiratory functions and could have a significant effect in alleviating disease conditions especially in children.

Probiotics therapy has been successfully tried with antibiotics associated diarrhoea as well as rotavirus diarrhoea, inflammatory disease and bowel syndromes, mucosal immunity and various other indications such as urogenital tract disorder, bacterial vaginosis, yeast vaginitis and also to maintain a healthy gut flora (as a prophylactic) in healthy individuals Most probiotic formulations are solid oral dosage forms. These are the probiotic powders (originally freeze dried or spray dried) mixed with excipients and formulated as per requirement to tablets, capsules, powder sachets, etc.

The probiotic requirement varies depending on the strain, product type, intended application and stability. According to Council for Agricultural Science and Technology the products often do not meet label claims with regard to the numbers and types of viable microbes present in the product, and do not deliver the quantity that needs to be consumed for a health benefit. The Council further recommends that manufacturers label the genus, species, and strain for each probiotic in a product, along with the number of viable cells of each probiotic strain that will remain up to the end of shelf-life. It is imperative that the products deliver requisite dose throughout the duration of the shelf-life and exhibit minimal batch to batch variation.

Accordingly, the product should amongst other aspects, comply with the following label requirements:

1) Genus and species identification, according to scientific nomenclature 2) Designate strain 3) Indicate viable count at the end of shelf-life 4) Specify storage conditions 5) Specify recommended dose based on the physiological effect desired.

Most probiotic formulations are provided as solid oral dosage forms. In general there is a need for a palatable liquid probiotic formulation which can be stored at ambient conditions without significant loss in probiotic activity over the recommended shelf life of the product. However until now there are no liquid probiotic formulations available in the market, which are palatable and can be stored at ambient storage conditions without significant loss in probiotic activity over the recommended shelf life of the product. This is because the Probiotics can grow using carbon and nitrogen sources in the formulation causing gas from fermentation and also giving rise to offensive odour and taste. The objective of the present invention is to meet this long felt need. Moreover, liquid probiotic formulations as compared to tablets will have better compliance in paediatric and geriatric populations.

Preparation of liquid probiotic formulations is highly desirable since elaborate and expensive processes such as freeze drying and microencapsulation used in the preparation of solid formulations are eliminated. The products are easy to administer. However preparation of liquid probiotic formulations which retain their cellular viability under ambient storage conditions over extended time periods to provide long shelf life to the product, poses following challenges.
1. The bacteria will start growing in the liquid media leading to metabolite production at ambient temperature.
2. It is known that the sugars act as substrates and enhance the metabolite production at ambient temperatures.
3. Gas producing probiotics will bulge the containers and produce offensive odours and taste.
4. Metabolite production will result in death of the probiotic strain and lower counts. Shelf life of such a formulation will be very short at ambient temperatures.

### DISCUSSION OF THE RELATED ART

“Enterogermina” produced and marketed by Sanofi is a liquid oral suspension of Bacillus clausi spores containing 2 billion spores in 5 ml saline. While this is a liquid suspension (formulation) it is not palatable and appealing in terms of taste, especially to children and more so to children with nausea associated with antibiotic associated diarrhoea.

WO2005017095 describes a liquid probiotic composition of E. coli M17. This is a suspension of culture free washed E. coli in 0.2-0.6% saline buffered to a pH of 6.5-6.8. The product needs to be stored between +2° C. to +8° C.

Patent application US20120171329 refers to a probiotic fruit juice drink composed of probiotic bacteria and a gas formation reducer chosen from acelora, pomegranate, cranberry, argnia, blackcurrant, blackthorn or elderberry extract only. The probiotic bacteria herein is chosen from Lactobacillus species, especially Lactobacillus plantarum. The product needs to be stored between 4-8° C. This composition is then further added to the other juices which are required to be made gas free. This limits the scope of invention to the specified gas reducer juice combinations only.

Patent application US20100086646 relates to fresh plant juice and or milk based food product comprising live probiotics and a dietary protonated weak acid with a pH between 3-4 which prevented false taste and gas in the product.

As cited in US20130295226, pH and temperature affect the organoleptic properties of the food product and the survival of the probiotic. These inventors used an acid adapted strain of L. casei and L. paracasei to overcome the above problems of pH and temperature limitations. This method is therefore restricted to specially adapted strains.

### SUMMARY OF THE INVENTION

It has been now found that liquid compositions comprising probiotic bacteria of the genera bacillus/lactobacillus, are stable at ambient storage conditions in the presence of sweeteners such as sugars which constitute at least 60% by weight of the liquid compositions comprising probiotic bacteria.

The present invention relates to stable liquid compositions comprising probiotic bacteria of the genera bacillus/lactobacillus, hydrophilic vehicles, solubilizers, pH modifiers, buffers, viscosity modifiers, preservatives, stabilizers and sweeteners. The said compositions retain their cellular viability under ambient storage conditions for at least two years as measured by the spores count test. The said compositions retain their cellular viability under accelerated aging test conditions for at least six months as measured by the spores count test. The compositions of the present invention are stable for use as probiotic syrup, in food, food supplement, and pharmaceutical applications.

The present invention also envisages a liquid composition comprising yeast and at least 60% sweetener by weight, and one more or more excipients selected from the group consisting of hydrophilic vehicles, solubilizer, pH modifier, buffer, viscosity modifier, preservatives, and stabilizers, wherein the said probiotic bacteria retains at least 75% of its cellular viability of the original value as measured by the spore count. The yeast of the invention is preferably saccharomyces boulardii.

### DETAILED DESCRIPTION OF THE INVENTION

The objectives of the present invention are accomplished and the shortcomings associated with the probiotic compositions of the prior art are overcome by the present invention as described below in the preferred embodiments.

The present invention encompasses liquid compositions of probiotic bacteria that are stable at ambient conditions. The bacteria of the invention is selected from the group consisting of genera bacillus.

In an embodiment the percentage of sweetener in the formulation is preferably 60%, 70% or 80%.

In an embodiment, the formulation of the invention comprises at least 60% sweetener, which does not promote the growth of bacterial spores.

In another embodiment of the invention, the probiotic compositions of the present invention have pH in the range 3.5 to 6.0.

In yet another embodiment the present invention provides probiotic compositions comprising 1) one or more Bacillus species in spore form, 2) a hydrophilic vehicle 3) a solubilizer which is a surfactant 4) pH modifiers and buffering agents 5) viscosity modifying agents 6) preservatives 7) stabilizers and 8) sweeteners

In yet another embodiment, the present invention also provides probiotic compositions comprising 1) one or more Lactobacillus species, 2) a hydrophilic vehicle 3) a solubilizer which is a surfactant 4) pH modifiers and buffering agents. 5) Viscosity modifying agents 6) preservatives 7) stabilizers and 8) sweeteners.

In another embodiment of the invention are provided probiotic compositions which when stored at ambient storage conditions for 24 months do not promote E coli growth.

In another embodiment of the invention, the present invention provides probiotic compositions wherein the pH of the composition when stored at ambient storage conditions for 24 months does not decrease by more than 0.1.

In another embodiment, the invention provides a liquid composition comprising probiotic bacteria which belongs to the genus Bacillus.

In another embodiment the invention provides a liquid composition comprising probiotic bacteria selected from Bacillus subtilus, Bacillus clausi and Bacillus coagulans.

In another embodiment the invention provides a liquid composition comprising a probiotic bacteria which belongs to the genus Lactobacillus.

In another embodiment the invention provides a liquid composition comprising a probiotic bacteria selected from lactobacillus acidophilus and lactobacillus casei

In another embodiment, the invention provides a liquid composition comprising probiotic bacteria which is a mixture of probiotic bacteria selected from the genus bacillus and lactobacillus.

In another embodiment, the invention provides a liquid composition comprising a probiotic bacteria and a sweetener.

In another embodiment, the invention provides a liquid composition comprising a probiotic bacteria and a sweetener wherein the sweetener is a sugar.

In another embodiment the invention provides a liquid composition comprising a probiotic bacteria and a sugar selected from sucrose, glucose, and fructose.

In another embodiment the invention provides a liquid composition comprising a probiotic bacteria and a sugar wherein the sugar is selected from a mixture of sucrose, glucose, and fructose.

In another embodiment the invention provides a liquid composition comprising a probiotic bacteria and a sweetener wherein the sweetener is a mixture of sugar and sugar alcohol.

In another aspect of the invention, the liquid compositions comprising probiotic bacteria, which are not stable in the presence of sugar alcohol alone, are stable in the presence of sugar alcohols and sugars.

In another embodiment the invention provides a liquid composition comprising a probiotic bacteria and a mixture of sugar and sugar alcohol, wherein the sugar alcohol is selected from mannitol, sorbitol and xylitol.

In another embodiment the invention provides a liquid composition comprising a probiotic bacteria and a sugar and sugar alcohol wherein the ratio of sugar to sugar alcohol ranges from 0.85 to 2.25.

In a preferred embodiment the compositions of the invention retain cellular viability under ambient storage conditions for at least two years for use as probiotic syrup in food, food supplement and pharmaceutical composition, particularly for health care.

The bacteriostatic preservative in the formulation is selected from the group consisting of bronidiol, a combination of methyl paraben and propyl paraben, sodium salt of parabens. Preferably, the preservative is bronidiol.

The present invention describes probiotic compositions having pH in the range 3.5 to 6.0. Citric acid serves as the pH modifier in the instant formulation.

The present invention also envisages a liquid composition comprising yeast and at least 60% sweetener by weight, and one more or more excipients selected from the group consisting of hydrophilic vehicles, solubilizer, pH modifier, buffer, viscosity modifier, preservatives, and stabilizers, wherein the said probiotic bacteria retains at least 75% of its cellular viability of the original value as measured by the spore count. The yeast of the invention is preferably saccharomyces boulardii.

The foregoing descriptions of specific embodiments of the present invention have been presented for purposes of illustration and description only. They are not intended to be exhaustive or to limit the present invention to the precise forms disclosed, and obviously many modifications and variations are possible in light of the above teaching.

### Example 1

Method of Preparation of Probiotic Composition

The compositions of the present invention are prepared as per the procedure described below: Water is heated in a vessel to 70° C. Vehicle and sweetener are added with stirring. After a homogenous solution is formed, the same is filtered to clarify and cooled to 45° C. The probiotic powder is added with stirring to form a homogenous solution. All other components of the formulation except the bacteriostatic preservative are added at this stage. Finally the bacteriostatic preservative, for example, Bronidiol is added and the volume of the composition is made up to 1000 ml using requisite quantity of water.

### Example 2

Method for the Determination of Spores Count
1. Take 1 gram of sample and suspend in 99 ml of saline+0.5% Tween 80 in an Erlenmeyer flask (This is 100 times dilution)
2. Keep it on a rotary shaker (200 rpm) at 37° C. for 30 mins.
3. Transfer 0.5 ml from the above flask to 4.5 ml of saline and carry out serial dilutions up to 10⁹
4. Surface spread 0.1 ml of the last 3 (10⁷, 10⁸, 10⁹) dilutions in duplicate on sterile Luria Bertani agar or Soybean casein Digest media (SCDM) agar plate using sterile spreader.
5. Incubate the plates at 37° C. and count the number of colony forming units.

Spore count CFU/gm=Number of colonies×Dilution factor×volume of sample used
For example If 10⁷ dilution plate gave 10 colonies, then
Spore count CFU/gm 10×10⁷×10=1.0×10⁹ cfu/ml or 5.0×10⁹ cfu/5 ml
Media composition

| 1) | Diluent | | |
|---|---|---|---|
| | Sodium chloride | 0.85 | gm |
| | Tween 80 | 0.5 | gm |
| | Distilled water | 100 | ml |
| 2) | Phenol red dextrose broth | | |
| | Phenol red dextrose broth (Hi media MO56) | 2.1 | gm |
| | Distilled water | 100 | ml |
| 3) | Soya bean casein digest medium (SCDM) | | |
| | Soya bean casein digest medium broth (Himedia MO11) | 3 | gm |
| | Agar powder (Himedia -RM026) | 2 | gm |
| | Distilled water | 100 | ml |
| 4) | Luria Bertani agar | | |
| | Tryptone (Casein hydrolysate) | 1 | gm |
| | Yeast extract | 0.5 | gm |
| | Sodium chloride | 0.5 | gm |
| | Distilled water | 100 | ml |
| | pH | 7.5 | |
| | Agar powder (Himedia -RM026) | 2 | gm |

For all spore count measurements to evaluate stability following conditions were used.

Ambient Storage Conditions

Temperature 30° C.±2° C., Humidity 75% RH±5% RH

RH=Relative Humidity

Storage Conditions for Accelerated Aging Test

Temperature 40° C.±2° C., Humidity 65% RH±5% RH

### Example 3

Formulation of the Probiotic Composition

| Sucrose | 600 | gm |
|---|---|---|
| B. subtilis | 2 × 10⁹/5 ml | |
| Flavour strawberry | 5 | gm |
| Colour FDC Red | 0.2 | gm |
| Bronidiol | 1 | gm |
| Water q.s. | 1000 | ml |

Results of Accelerated Stability Test

| | Months | | | | |
|---|---|---|---|---|---|
| | 0 | 1 | 2 | 3 | 6 |
| Spore count 10⁹/5 ml | 3.8 | 3.1 | 2.85 | 4.1 | 3.7 |

Results of Stability Test Under Ambient Storage Conditions

| Months | 0 | 1 | 2 | 3 | 6 | 12 | 18 | 24 |
|---|---|---|---|---|---|---|---|---|
| Spore count 10⁹/5 ml | 2.7 | 2.4 | 3.6 | 2.6 | 3.1 | 2.5 | 2.2 | 2.6 |
| pH | 3.5 | 3.5 | 3.4 | 3.6 | 3.5 | 3.4 | 3.5 | 3.6 |

The comparative results show that results of stability test under ambient storage conditions are adequately simulated by the results of the accelerated stability test.

### Example 4

Formulation of the Probiotic Composition with Change in Bacterial Concentration

| Sucrose | 600 | gm |
|---|---|---|
| B. subtilis | 0.2 × 10⁹/5 ml | |
| Flavour strawberry | 5 | gm |
| Colour FDC Red | 0.2 | gm |
| Bronidiol | 1 | gm |
| Water q.s. | 1000 | ml |
| Sucrose | 600 | gm |
| B. subtilis | 10 × 10⁹/5 ml | |
| Flavour strawberry | 5 | gm |
| Colour FDC Red | 0.2 | gm |
| Bronidiol | 1 | gm |
| Water q.s. | 1000 | ml |

### Example 5

Formulation of the Probiotic Composition with Change in Sweetener

| High fructose corn syrup | 780 | gm |
|---|---|---|
| B. subtilis | 2 × 10⁹/5 ml | |
| Flavour strawberry | 5 | gm |
| Colour FDC Red | 0.2 | gm |
| Bronidiol | 1 | gm |
| Water q.s. | 1000 | ml |
| Sucrose + High fructose corn syrup | 300 gm + 390 gm | |
| B. subtilis | 2 × 10⁹/5 ml | |
| Flavour strawberry | 5 | gm |
| Colour FDC Red | 0.2 | gm |
| Bronidiol | 1 | gm |
| Water q.s. | 1000 | ml |

### Example 6

| Sucrose | 600 | gm |
|---|---|---|
| Sorbitol | 150 | gm |
| B. subtilis | 2 × 10⁹/5 ml | |
| Flavour strawberry | 5 | gm |
| Colour FDC Red | 0.2 | gm |
| Bronidiol | 1 | gm |
| Water q.s. | 1000 | ml |
| Sucrose | 600 | gm |
| Glycerol | 50 | gm |
| B. subtilis | 2 × 10⁹/5 ml | |
| Flavour strawberry | 5 | gm |
| Colour FDC Red | 0.2 | gm |
| Bronidiol | 1 | gm |
| Water q.s. | 1000 | ml |

### Example 7

Formulation of the Probiotic Composition with pH Modifier

| Sucrose | 600 | gm |
|---|---|---|
| B. subtilis | 2 × 10⁹/5 ml | |
| Flavour strawberry | 5 | gm |
| Colour FDC Red | 0.2 | gm |
| Bronidiol | 1 | gm |
| Water q.s. | 1000 | ml |

| | |
|---|---|
| Citric acid | 0.1% |

### Example 8

Formulation of the Probiotic Composition with Change in Preservative

| Sucrose | 600 | gm |
|---|---|---|
| B. subtilis | 2 × 10⁹/5 ml | |
| Flavour strawberry | 5 | gm |
| Colour FDC Red | 0.2 | gm |
| Methyl Paraben + Propyl Paraben sodium salt | 0.075% + 0.075% | |
| Water q.s. | 1000 | ml |
| Sucrose | 600 | gm |
| B. subtilis | 2 × 10⁹/5 ml | |
| Flavour strawberry | 5 | gm |
| Colour FDC Red | 0.2 | gm |
| Sodium Benzoate | 0.1% | |
| Water q.s. | 1000 | ml |

### Example 9

Formulation of the Probiotic Composition with a Thickener

| Sucrose | 600 | gm |
|---|---|---|
| B. subtilis | 2 × 10⁹/5 ml | |
| Flavour strawberry | 5 | gm |
| Colour FDC Red | 0.2 | gm |
| Bronidiol | 1 | gm |
| Water q.s. | 1000 | ml |
| Methyl cellulose | 0.5% | |
| Sucrose | 600 | gm |
| B. subtilis | 2 × 10⁹/5 ml | |
| Flavour strawberry | 5 | gm |
| Colour FDC Red | 0.2 | gm |
| Bronidiol | 1 | gm |
| Water q.s. | 1000 | ml |

| | |
|---|---|
| Carboxy methyl cellulose | 1% |

In the following examples only the contents of the probiotic bacteria and the sugar/sugar alcohol are mentioned. Other ingredients are as per example 3

### Example 10

| | |
|---|---|
| Sucrose | 300 gm |
| Sorbitol | 350 gm |

Results of Accelerated Stability Test

| | Months | | | | |
|---|---|---|---|---|---|
| | 0 | 1 | 2 | 3 | 6 |
| Spore count 10⁹/5 ml | 2.7 | 2.4 | 3.6 | 2.6 | 3.1 |

### Example 11

| | |
|---|---|
| Sorbitol | 650 gm |

Composition not Stable

### Example 12

| | |
|---|---|
| Sucrose | 250 gm |
| Mannitol | 400 gm |

Results of Accelerated Stability Test

| | Months | | | | |
|---|---|---|---|---|---|
| | 0 | 1 | 2 | 3 | 6 |
| Spore count 10⁹/5 ml | 3.1 | 2.4 | 2.9 | 3.6 | 3.4 |

### Example 13

| | |
|---|---|
| Mannitol | 650 gm |

Composition not Stable

### Example 14

| | |
|---|---|
| Sucrose | 550 gm |
| Glycerol | 100 gm |
| B. subtilis | 6.47 × 10⁹/5 ml |

Results of Accelerated Stability Test

| | Months | | | | |
|---|---|---|---|---|---|
| | 0 | 1 | 2 | 3 | 6 |
| Spore count 10⁹/5 ml | 3.8 | 3.1 | 2.85 | 4.1 | 3.7 |

### Example 15

| | |
|---|---|
| Glycerol | 650 gm |
| B. subtilis | 6.2 × 10⁹/5 ml |

Composition not Stable

### Example 16

| | |
|---|---|
| Sucrose | 350 gm |
| Glucose | 100 gm |
| Sorbitol | 200 gm |
| B. subtilis | 6.4 × 10⁹/5 ml |

Results of Accelerated Stability Test

| | Months | | | | |
|---|---|---|---|---|---|
| | 0 | 1 | 2 | 3 | 6 |
| Spore count 10⁹/5 ml | 2.7 | 2.4 | 3.6 | 2.6 | 3.1 |

### Example 17

| | |
|---|---|
| Sucrose | 250 gm |
| Glucose | 250 gm |
| Glycerol | 150 gm |
| B. subtilis | 5.8 × 10⁹/5 ml |

Results of Accelerated Stability Test

| | Months | | | | |
|---|---|---|---|---|---|
| | 0 | 1 | 2 | 3 | 6 |
| Spore count 10⁹/5 ml | 3.1 | 2.4 | 2.9 | 3.6 | 3.4 |

### Example 18

In this and all subsequent examples the liquid probiotic composition as in example 3 is used. The bacteria species and content is varied as indicated

Species Bacillus clausi: Content 3.8×10⁹/5 ml

Results of Accelerated Stability Test

| | Months | | | | |
|---|---|---|---|---|---|
| | 0 | 1 | 2 | 3 | 6 |
| Spore count 10⁹/5 ml | 3.8 | 3.5 | 2.9 | 3.1 | 3.2 |
| pH | 3.6 | 3.5 | 3.6 | 3.6 | 3.6 |

Results of Stability Test Under Ambient Conditions

| Months | 0 | 1 | 2 | 3 | 6 | 12 | 18 | 24 |
|---|---|---|---|---|---|---|---|---|
| Spore count 10⁹/5 ml | 3.8 | 3.1 | 3.3 | 3.1 | 3.2 | 3.6 | 3.7 | 3.5 |
| pH | 3.6 | 3.6 | 3.5 | 3.6 | 3.5 | 3.5 | 3.6 | 3.5 |

### Example 19

Species Bacillus coagulans: Content 2.4×10⁹/5 ml

Results of Accelerated Stability Test

| | Months | | | | |
|---|---|---|---|---|---|
| | 0 | 1 | 2 | 3 | 6 |
| Spore count 10⁹/5 ml | 2.4 | 2.2 | 2.1 | 2.4 | 2.2 |
| pH | 3.6 | 3.6 | 3.6 | 3.5 | 3.6 |

Results of Stability Test Under Ambient Conditions

| Months | 0 | 1 | 2 | 3 | 6 | 12 | 18 | 24 |
|---|---|---|---|---|---|---|---|---|
| Spore count 10⁹/5 ml | 2.4 | 2.2 | 2.1 | 2.4 | 2.2 | 2.6 | 2.1 | 2.5 |
| pH | 3.6 | 3.6 | 3.6 | 3.6 | 3.5 | 3.5 | 3.6 | 3.6 |

### Example 20

Species: Lactobacillus acidophilus Content 2.8×10⁹/5 ml

Results of Accelerated Stability Test

| | Months | | | | |
|---|---|---|---|---|---|
| | 0 | 1 | 2 | 3 | 6 |
| Spore count 10⁹/5 ml | 2.4 | 2.2 | 2.1 | 2.4 | 2.2 |

### Example 21

Species: Bacillus subtilis Content 3.8×10⁹/5 ml+Lactobacillus casei Content 3.4×10⁹/5 ml

Results of Accelerated Stability Test

| | Months | | | | |
|---|---|---|---|---|---|
| | 0 | 1 | 2 | 3 | 6 |
| Bacillus subtilis Spore | 3.8 | 3.1 | 2.85 | 4.1 | 3.7 |
| count 10⁹/5 ml | | | | | |
| Lactobacillus casei | 3.4 | 3.2 | 3.1 | 3.6 | 3.1 |
| Spore count 10⁹/5 ml | | | | | |
| pH | 3.5 | 3.5 | 3.5 | 3.5 | 3.6 |

We claim:

1. A liquid composition comprising probiotic bacteria and at least 60% sweetener weight by volume, wherein the said probiotic bacteria retains at least 75% of its cellular viability of the original value as measured by the spore count.

2. A liquid composition comprising probiotic bacteria, at least 60% sweetener weight by volume, and one more or more excipients selected from the group consisting of hydrophilic vehicles, solubilizer, pH modifier, buffer, viscosity modifier, preservatives, and stabilizers, wherein the said probiotic bacteria retains at least 75% of its cellular viability of the original value as measured by the spore count.

3. The liquid composition comprising a probiotic bacteria as claimed in claim 1, wherein the cellular viability as measured by the spore count is retained to at least 75% of the original value, when the said liquid composition is stored at ambient storage conditions for a period of at least 6 to 24 months.

4. The liquid composition comprising a probiotic bacteria as claimed in claim 1, wherein the said probiotic bacteria belongs to the genus Bacillus, genus Lactobacillus or a mixture of both.

5. The liquid composition comprising a probiotic bacteria as claimed in claim 4, wherein the said probiotic bacteria is selected from the species Bacillus subtilus, Bacillus clausi and Bacillus coagulans.

6. The liquid composition comprising a probiotic bacteria as claimed in claim 4, wherein the said probiotic bacteria is selected from the species lactobacillus acidophilus and lactobacillus casei.

7. The liquid composition comprising a probiotic bacteria as claimed in claim 1, wherein the spore count of the composition prepared is in the range 0.2×10⁹ cfu/5 ml to 10×10⁹ cfu/5 ml.

8. The liquid composition comprising a probiotic bacteria as claimed in claim 1, wherein the sweetener is a sugar.

9. The liquid composition comprising a probiotic bacteria as claimed in claim 8, wherein the sugar is selected from the group consisting of sucrose, glucose, and fructose.

10. The liquid composition comprising a probiotic bacteria as claimed in claim 1, wherein the sweetener is a mixture of sugar and sugar alcohol at ratio of sugar to sugar alcohol in the range of 0.85 to 2.25.

11. The liquid composition comprising a probiotic bacteria as claimed in claim 10, wherein the sugar alcohol is mannitol, sorbitol, xylitol or a mixture thereof.

12. A liquid composition comprising yeast, preferably, saccharomyces boulardii and at least 60% sweetener by weight, and one more or more excipients selected from the group consisting of hydrophilic vehicles, solubilizer, pH modifier, buffer, viscosity modifier, preservatives, and stabilizers, wherein the said probiotic bacteria retains at least 75% of its cellular viability of the original value as measured by the spore count.
